(19) United States
(12) Patent Application Publication
(10) Pub. No.: US 2026/0165613 A1
(43) Pub. Date: Jun. 18, 2026

(54) ANALYTE SENSOR

(71) Applicant: GLUCORX TECHNOLOGIES LIMITED, Dingwall (GB)

(72) Inventors: Cerys Rohann MURRAY-SCOTT, Dingwall (GB); Andrew MCCULLOCH, Framlingham (GB); James MOFFATT, Dingwall (GB); James Iain RODGERS, Dingwall (GB)

(21) Appl. No.: 19/124,801

(22) PCT Filed: Sep. 29, 2023
(86) PCT No.: PCT/EP2023/077166

§ 371 Date: Apr. 25, 2025

(30) Foreign Application Priority Data
Oct. 26, 2022 (GB) 2215849.7

(51) Int. Cl. A61B 5/145

(52) U.S. Cl. CPC A61B 5/14532; A61B 5/14503; A61B 2562/12

(57) ABSTRACT

A skin-implantable analyte sensor for use with an analyte monitoring system, the analyte sensor comprising: a tail portion configured for insertion into a user's skin, the tail portion having a proximal end and a distal end; a lateral portion extending in a lateral direction from the tail portion, the tail portion and the lateral portion defining a plane; and a contact portion having one or more conductive contact pads thereon, wherein, during use, the contact portion extends from the lateral portion in a direction out of and perpendicular to the plane; wherein a length of the lateral portion in the lateral direction is greater than a length of the contact portion in the lateral direction.

### FIELD OF INVENTION

The present disclosure relates to a skin-implantable analyte sensor for use with an analyte monitoring system, and a method of manufacturing such a sensor.

### BACKGROUND

Traditionally, monitoring blood glucose levels of a patient involved a process where a finger prick blood test obtained a small drop of blood that was placed on a test strip that inserted into glucometer. The glucometer read the strip and provided a digital reading of the individual's blood sugar level.

Recently, finger prick blood tests have been replaced by insertable (implantable), in vivo, analyte sensors that are inserted into the skin of the patient where they remain at all times, enabling substantially continuous measurements to be taken, which is advantageous compared to finger prick tests that provide only snapshot readings at a small number of times a day. These types of implantable analyte sensors are typically coupled to a sensor module having a housing placed on the surface of the patient's skin. For example, the rearwardly protruding part of the analyte sensor is inserted into the skin-facing surface of the housing the sensor module, where it is coupled inside the sensor module to control electronics. This ensures only the sensor module itself is visibly exposed to the outside environment and the entry point of the insertable analyte sensor in the skin is at least partially protected underneath the sensor module. The sensor module control electronics process measurement signals from the inserted sensor and transmit any relevant information to, for example, the patient's smartphone or other mobile device. These types of systems are sometimes known as continuous analyte monitoring systems.

Typically, continuous analyte monitoring systems require the analyte sensor to be replaced at predetermined intervals and this may require the patient to insert the analyte sensor themselves in an unsupervised environment. In order to simplify this process, known analyte monitoring systems are provided with an inserter device which applies a predetermined amount of force to the insertable analyte sensor to safely insert it into the patient's skin, and at the same time to position any accompanying sensor module at the surface of the patient's skin. EP2393417B1 proposes a continuous analyte monitoring system.

In known continuous analyte monitoring systems, such as that of EP2393417B1, the sensor module is initially inside of the inserter device. An adhesive pad or patch (sometimes referred to as an epidermal support patch) is provided on the skin-facing end of the inserter device. During use, the inserter device inserts the analyte sensor into the skin, places the sensor module onto the adhesive pad which is thereby secured to the patient's skin.

Analyte sensors of the type used in continuous analyte monitoring systems such as that of EP2393417B1 have a portion that is inserted into the skin of the user and a portion perpendicular thereto during use that is coupled to the sensor module of the system with conductive contact pads.

Analyte sensors are typically flat immediately after manufacture as they are made by stamping the shape of the sensor out of a flat sheet of plastic material. Upon assembly of the continuous analyte monitoring system, the stamped shape is folded along a predetermined line to deform the portion with the contact pads thereon to make it perpendicular to the tail portion and ready for insertion into the sensor module.

A problem of existing analyte sensors is that their manufacture results in large amounts of waste plastic. They also break easily for example during folding.

An improved analyte sensor is desired.

### SUMMARY

In general terms, a first aspect of the present disclosure is directed to an analyte sensor for an analyte monitoring system that has a longer portion that extends laterally from a tail portion than known sensors. The longer lateral portion facilitates ways to make and deform the analyte sensor into the intended configuration for use that are less likely to result in damage to the sensor compared to known systems. As a result, there are fewer defective sensors in each production run.

Thus, according to a first aspect of the disclosure, there is provided a skin-implantable analyte sensor for use with an analyte monitoring system, the analyte sensor comprising: a tail portion configured for insertion into a user's skin, the tail portion having a proximal end and a distal end; a lateral portion extending in a lateral direction from the tail portion, the tail portion and the lateral portion defining a plane; and a contact portion having one or more conductive contact pads thereon,, wherein, during use, the contact portion extends from the lateral portion in a direction out of and perpendicular to the plane, wherein a length of the lateral portion in the lateral direction is greater than a length of the contact portion in the lateral direction.

As described above, a problem that occurs during manufacture of analyte sensors is that the conductive contact pads made of conductive material deposited in a predetermined position on the shape are fragile and easily damaged when the shape of the analyte sensor is stamped or otherwise removed from the sheet it is manufactured from. The same concern applies to other parts of the analyte sensor such as any particularly thin joins between any of the portions of the analyte sensor that might otherwise snap or break when the sensor is deformed to position the contact portion perpendicularly to and extending out of the plane of the sensor during use. Advantageously, increasing the length of the lateral portion moves the contact portion (and thus the contact pads) away from the edge of the tail portion where a bending or cutting force is applied by the stamp used to remove the analyte sensor from the sheet. Thus, when the lateral portion is greater in length in the lateral direction than the contact portion, the contact portion is spaced apart from the tail portion to avoid damage to the contact pads and/or any thin join between the contact portion and the lateral portion that might otherwise occur during manufacture. Further, it avoids any stray conductive material being deposited on the tail portion, thereby reducing the number defects in each manufacturing run. Finally, as is described below, a longer lateral portion also facilitates the use of alternative deformation techniques that are not possible with existing sensors that have short lateral portions.

Optionally, during use, the lateral portion comprises a twisted section and, optionally, the twisted section may comprise a 90-degree twist.

Advantageously, a longer lateral portion allows a twist to be used to deform the sensor to bring the contact portion into its perpendicular position relative to the plane of the sensor instead of using a bend or fold deformation used in known sensors. This is because the short lateral portion of known sensors is unable to sustain the steep twist pitch or angle to achieve the full 90-degree twist over its short length without snapping. Instead, by extending the lateral portion out to be longer than the contact portion, the twist pitch or angle no longer has to be as steep as the twist can be achieved across a much larger length of the lateral portion. As a result, the lateral portion is better able to accommodate the twist to 90 degrees without snapping.

Further, a twist instead of a bend or fold is particularly advantageous because the twist's restorative force (back in the direction of the plane) is typically stronger than a bend or a fold. This stronger force can be used to bias more effectively the contact portion into contact with corresponding contact pads on a PCB of a sensor module with which the sensor is being used compared to known sensors.

Finally, a gentle twist along a long lateral portion reduces how extremely any conductive traces have to be bent or folded which might otherwise cause the conductive link to break. Thus, the twist reduces the risk that conductive traces are broken compared to a bend or fold of known sensors.

Optionally, the analyte sensor comprises one or more perforations between the lateral portion and the contact portion, and optionally, prior to use, the contact portion is configured to be folded relative to the lateral portion along the one or more perforations.

Advantageously, a longer lateral portion may additionally or alternatively to the twist facilitate the introduction of perforations between the lateral portion and contact portion. Perforating the join between the lateral portion and the contact portion allows the contact portion to be folded or bent to be perpendicular to the plane defined by the tail portion and the lateral portion more easily and with a reduced risk of breakage compared to known sensors because the contact portion is joined to the lateral portion at a plurality of locations (between each perforation). Thus, even if one join section snaps, the others ensure the contact portion does not break off. In other words, the perforation provides for the contact portion and the lateral portion being joined at or along a plurality of points or edges (namely between the perforation holes) and the problems of an easily breakable thin join between the contact portion and the lateral portion that exist in known systems are eliminated.

Optionally, during use, a length of at least part of the lateral portion in the proximal direction is greater than a thickness of the contact portion in the proximal direction.

Implanting analyte sensors requires the assistance of a cannula or other sharp to pierce a user's skin and to keep the analyte sensor straight as it is inserted. Once the analyte sensor is inserted and coupled with a sensor module, it needs to be held in a position where the tail portion remains perpendicular to a user's skin. A problem of known analyte sensors such as that of EP2393417B1 is that they may be unstable or rotate when held in the sensor module. Advantageously, providing a lateral portion that is not only longer than the contact portion, but which also has a substantial extent in the proximal direction provides for a more stable analyte sensor. This is because the lateral portion has an increased surface area in the plane in the proximal direction which can more easily be gripped or held by the sensor module and at a greater distance from the tail portion to better resist any rotational forces about the tail portion or about other axes. Thus, the lateral portion functions as a positioning or alignment lip configured for engagement with a support structure of a sensor module or tab to avoid rotation and ensure correct placement of the analyte sensor in a cannula and in a sensor module.

Optionally, the conductive contact pads are provided at an edge of the contact portion.

During use, the analyte sensor may be configured to be held in a shaped slot of the sensor module and the conductive contact pads may be configured to contact a corresponding contact of a printed circuit board (PCB) of the sensor module. In known analyte sensors such as that of EP2393417B1, contact pads are provided away from any edges, that is, not contacting the edges of the analyte sensor. This can make it difficult to exactly deposit conductive material of contact pads of an analyte sensor in the correct position that would align with those of a PCB during as manufacturing tolerances often result in the exact positioning of the contact pads during a deposition step being imprecise. Instead, advantageously, providing the contact pads on an edge of the contact portion, i.e. where the stamp cuts or presses the analyte sensor shape out of a sheet of material, enables the conductive material deposition step to be less accurate (and thus cheaper) as long as the contact portion edge has at least some conductive material thereon. In this way, the edge of the contact portion can used to as an alignment guide when the sensor is positioned in a corresponding slot of the sensor module. As a result of using the edge as a guide, the risk of any short circuits from incorrect alignment of contact pads is substantially reduced.

Optionally said edge on which the contact pads are provided may be parallel to the lateral direction.

Advantageously, this allows the extent of the contact portion extending out of the plane during use be reduced in size as the contact pads are spread out along the length of the contact portion in the lateral direction rather than perpendicular thereto.

Optionally, the contact portion comprises a deformable finger portion having one of said conductive contact pads thereon.

Advantageously, providing a deformable portion on the contact portion allows the contact portion to be used as a switch, for example, as a momentary switch, to switch on and otherwise activate any circuitry of a sensor module with which the sensor is intended to be used. This approach is particularly advantageous as it ensures that a sensor module cannot switch on until an analyte sensor of the present disclosure is positioned therein and the deformable portion (for example a finger-like portion at an edge of the contact portion), thereby preventing any batteries being run down prematurely.

Optionally, the tail portion comprises a sensing layer electrically coupled to the one or more conductive contact pads by one or more conductive traces extending along a surface of the tail portion, a surface of the lateral portion, and a surface of the contact portion.

Advantageously, the traces allow a measured signal from a sensing end of the analyte sensor to be communicated to the one or more contact pads on the contact portion.

Optionally, prior to use, the contact portion is in the plane and extends from the lateral portion in a distal direction towards the distal end.

Advantageously, extending the contact portion in the distal direction results a shape that can be arranged more efficiently on a plastic sheet during manufacture to thereby reduce waste. For example, the contact portion of the analyte sensor before it is folded may extend back in the direction of a distal end of the tail portion to provide a paperclip-like or hanging flag like shape. The number of such analyte sensor shapes that can thereby fit onto a sheet during manufacture is increased and the total amount of waste plastic is reduced. Alternatively, optionally, prior to use, the contact portion is in the plane and extends from the lateral portion in a proximal direction away from the proximal end.

Optionally, the lateral portion extends from the tail portion at the proximal edge of the proximal end of the tail portion in said plane defined by the tail portion and the lateral portion.

As described above, during insertion of the analyte sensor by cannula, there is a risk that the analyte sensor may become misaligned or rotate. To avoid this, the lateral portion may be configured to engage a corresponding slot or holding shape in the cannula used to insert the analyte sensor. Advantageously, by providing the lateral portion at the very end of the tail portion (i.e. at the proximal edge of the proximal end), not only can the lateral portion effectively engage a corresponding slot on the cannula but it also ensures there are no fragile, thinner, parts of the analyte sensor that extend beyond this proximal edge. As described above thin parts have a far higher risk of breaking or snapping when the analyte sensor is bent and positioned in a sensor module. Thus, providing the lateral portion at the very end of the tail portion ensures that there are no such thin, fragile parts that remain exposed at the end of the tail portion.

Optionally, the skin-implantable analyte sensor may be configured as a blood-glucose analyte sensor.

Whilst it is envisaged that the analyte sensor of the present disclosure is a blood glucose sensor. It is also envisaged that the sensor may be used to measure other analytes.

Optionally, the analyte sensor may comprise an aperture, opening or eyelet configured to receive a protrusion of the housing, to thereby positionally secure the analyte sensor within the housing. A shape of the eyelet may be e.g. a circle or ellipse, or may be a shape comprising one or more corners or otherwise pointed ends (such as an oval or triangle).

In general terms, according to a second aspect of the disclosure, there is provided an analyte sensor wherein, prior to use, the shape of the sensor is such that the contact portion extends back towards the distal end of the tail portion to provide a much more compact shape in the sensor plane. This facilitates a much more efficient arrangement of multiple sensor shapes on a sheet during manufacture to reduce plastic wastage.

Thus, according to the second aspect, there is provided a skin-implantable analyte sensor for use with an analyte monitoring system, the analyte sensor comprising:
a tail portion configured for insertion into a user's skin, the tail portion having a proximal end and a distal end; a lateral portion extending in a lateral direction from the tail portion, the tail portion and the lateral portion defining a plane; and a contact portion having one or more conductive contact pads thereon, wherein, during use, the contact portion extends from the lateral portion in a direction out of and perpendicular to the plane, and wherein, prior to use, the contact portion is in the plane and extends from the lateral portion in a distal direction towards the distal end.

Advantageously, as described above, extending the contact portion in the distal direction results a shape that can be arranged more efficiently on a plastic sheet during manufacture to thereby reduce waste. For example, the contact portion of the analyte sensor before it is folded may extend back in the direction of a distal end of the tail portion to provide a paperclip-like or hanging flag like shape. The number of such analyte sensor shapes that can thereby fit onto a sheet during manufacture is increased and the total amount of waste plastic is reduced.

Optionally, the lateral portion comprises a shoulder portion at a lateral edge, wherein, prior to use, the contact portion extends in a distal direction from a distal edge of the shoulder portion of the lateral portion, and wherein, the analyte sensor is configured to be folded along a line between the shoulder portion and the contact portion.

As described above, the closer the contact portion is to the tail portion, the higher the risk that the manufacturing process of stamping out the analyte sensor may damage the contact portion. Advantageously, providing a shoulder portion at an edge of the lateral portion from which the contact portion extends allows the contact portion to be positioned further away from the tail portion, thereby reducing the risk of damage to the contact portion or tail portion during manufacture.

Optionally, the skin-implantable analyte sensor may be configured as a blood-glucose analyte sensor.

Whilst it is envisaged that the analyte sensor of the present disclosure is a blood glucose sensor. It is also envisaged that the sensor may be used to measure other analytes.

Optionally, the contact portion comprises a deformable finger portion having one of said conductive contact pads thereon.

Advantageously, providing a deformable portion on the contact portion allows the contact portion to be used as a switch, for example, as a momentary switch, to switch on and otherwise activate any circuitry of a sensor module with which the sensor is intended to be used. This approach is particularly advantageous as it ensures that a sensor module cannot switch on until an analyte sensor of the present disclosure is positioned therein and the deformable portion (for example a finger-like portion at an edge of the contact portion), thereby preventing any batteries being run down prematurely.

Optionally, the analyte sensor may comprise an aperture, opening or eyelet configured to receive a protrusion of the housing, to thereby positionally secure the analyte sensor within the housing. A shape of the eyelet may be e.g. a circle or ellipse, or may be a shape comprising one or more corners or otherwise pointed ends (such as an oval or triangle).

According to a third aspect of the disclosure, there is provided a method of manufacturing a skin-implantable analyte sensor of any preceding claim, the method comprising: providing a sheet of plastic material; depositing a conductive material on the sheet to form one or more conductive contact pads; and stamping a shape out of the sheet to form said skin-implantable analyte portion, the shape comprising: a tail portion having a proximal end and a distal end; a lateral portion extending in a lateral direction from the tail portion, the tail portion and the lateral portion defining a plane; and a contact portion having said one or more conductive contact pads thereon, wherein a length of the lateral portion in the lateral direction is greater than a length of the contact portion in the lateral direction.

Optionally, the method comprises twisting the lateral portion to cause the contact portion to extend out of the plane.

Optionally, the method comprises perforating the analyte sensor between the lateral portion and the contact portion.

Optionally, the method comprises folding the analyte sensor along the perforations to cause the contact portion to extend out of the plane.

It will be appreciated that the above described advantages of the first aspect of the disclosure apply equally to the corresponding features of the second aspect of the disclosure.

### BRIEF DESCRIPTION OF THE DRAWINGS

These and other aspects will now be described, by way of example only, with reference to the accompanying figures in which:

FIG. 1a illustratively shows an analyte sensor according to the present disclosure in a configuration during use.

FIG. 1b illustratively shows an alternative view of the analyte sensor of FIG. 1a.

FIG. 1c illustratively shows the analyte sensor of FIG. 1a in a configuration prior to use.

FIG. 1d illustratively shows an array of the analyte sensors of FIG. 1c.

FIG. 2a illustratively shows an analyte sensor according to the present disclosure prior to use.

FIG. 2b illustratively shows the analyte sensor of FIG. 2a in a configuration during use.

FIG. 2c illustratively shows an array of the analyte sensors of FIG. 2a.

FIG. 3a illustratively shows an analyte sensor according to the present disclosure prior to use.

FIG. 3b illustratively shows the analyte sensor of FIG. 3a in a configuration during use.

FIG. 3c illustratively shows an array of the analyte sensors of FIG. 3a.

FIG. 4 illustratively shows an analyte sensor of the present disclosure positioned in a sensor module of an analyte monitoring system.

FIG. 5 illustratively shows a flowchart of a method of the present disclosure.

FIG. 7a illustratively shows an analyte sensor according to the present disclosure.

FIG. 7b illustratively shows the analyte sensor of FIG. 8a from a different angle.

### DETAILED DESCRIPTION OF THE DRAWINGS

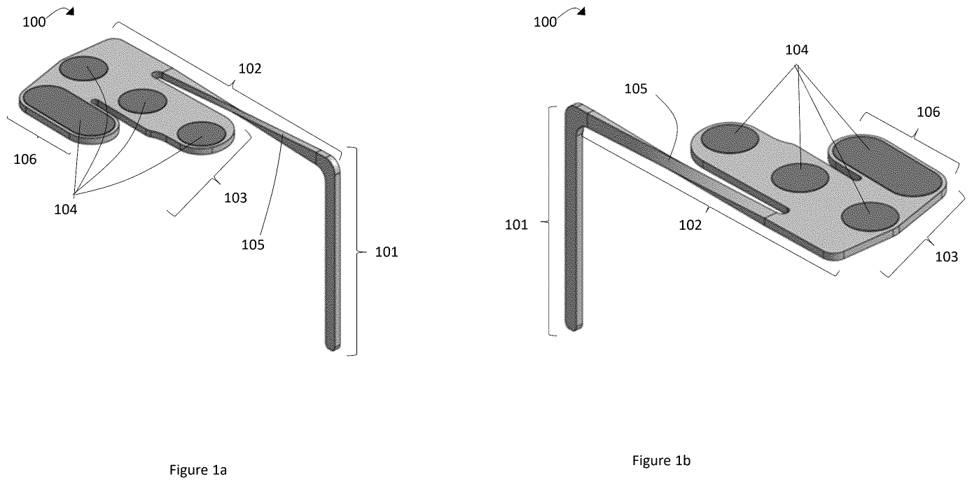

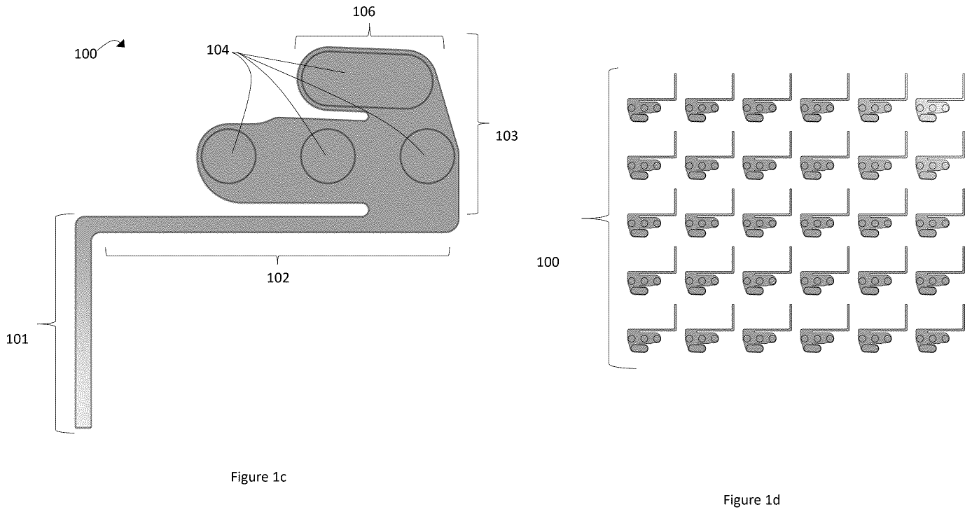

FIGS. 1a and 1b illustratively shows different views of an analyte sensor 100 according to the present disclosure. The analyte sensor 100 comprises a tail portion 101, a lateral portion 102 and a contact portion 103. The tail portion 101 and lateral portion 102 define a plane and the lateral portion 102 extends from a proximal end of the tail portion 101 in the plane. The contact portion 103 has thereon a plurality of contact pads 104 comprising an electrically conductive material. The contact pads 104 are electrically coupled to conductive traces (not shown) that extend from the contact pads 104 along the lateral portion 102 and the tail portion 101 to the distal end of the tail portion 101 to a sensing layer (not shown). The sensing layer may comprise an electron transfer agent but other sensing layers for implantable analyte sensors known to the skilled person are also envisaged.

The contact portion 103 extends from the lateral portion 102 out of the plane defined by the lateral portion 102 and the tail portion 101, in a direction perpendicular to that plane.

The lateral portion 102 has an extent in the lateral direction that is greater than the extent of the contact portion 103 in the lateral direction and this enables a twist to be provided in the lateral portion 102 without snapping or otherwise breaking it.

In order to allow the contact portion 103 to be perpendicular to the plane, the lateral portion 102 comprises a twisted section 105 along a portion of the length of the lateral portion 102. The twisted section in the sensor of FIGS. 1a and 1 b provides a 90 degree twist. It will be appreciated that this gentle twist as opposed to a sharp bend or fold means the mechanical strain on the plastic material of the lateral portion 102 and, on any conductive traces on a surface thereof, is substantially reduced. As a result, both the lateral portion 102 and any conductive traces thereon are less likely to break when the contact portion 103 is positioned in the perpendicular configuration for use.

The analyte sensor 100 in FIGS. 1a and 1b further comprises a deformable finger 106 that may be used as part of a switch, such as a temporary switch, to switch on a sensor module with which the sensor 100 is intended to be used. For example, a user causing applying a force may deform the deformable finger 106 in a direction out of the plane of the contact portion causing a contact pad 104 thereon to come into contact with a corresponding contact pad of a PCB of a sensor module (not shown) to switch on the sensor module.

FIG. 1c shows the analyte sensor 100 of FIGS. 1a and 1b in a flat configuration prior to use and prior to the introduction of the twist on the lateral portion. This is the configuration the sensor 100 is in when it is stamped or cut from a sheet of plastic during manufacture.

FIG. 1d shows a plurality of the analyte sensors 100 of FIG. 1c in an array before they have been stamped or cut from a sheet of plastic during manufacture. The array in FIG. 1d is shown to have eight sensors arranged therein, but other array sizes and numbers are also envisaged.

In the example of FIGS. 1c and 1d, the contact portion 103 extends upwards from a proximal side of the lateral portion 102 beyond the height of the proximal end of the tail portion 101. However, it is also envisaged that the contact portion 103 may instead extend from the distal side of the lateral portion 102. In this case, the overall footprint of the sensor before twisting is smaller and is accordingly able to be packed more densely in an array on a plastic sheet during manufacture compared to the example shown in FIGS. 1c and 1d.

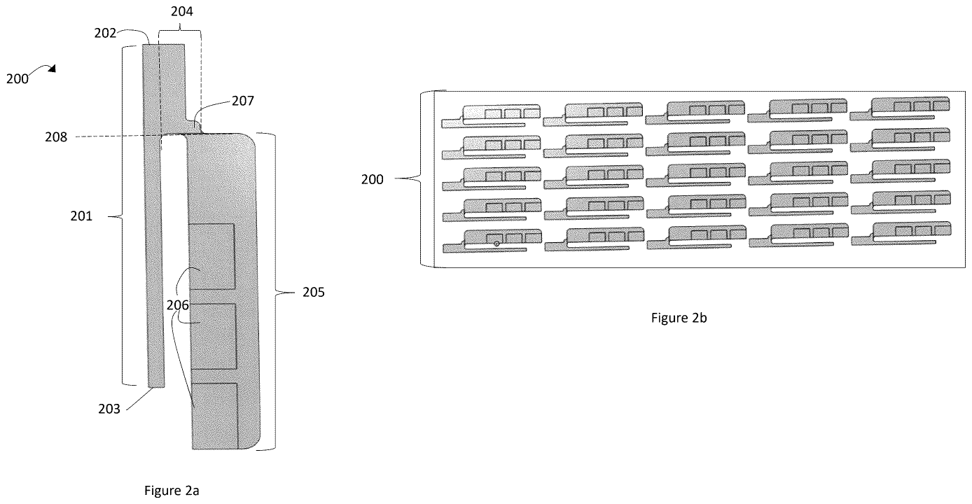

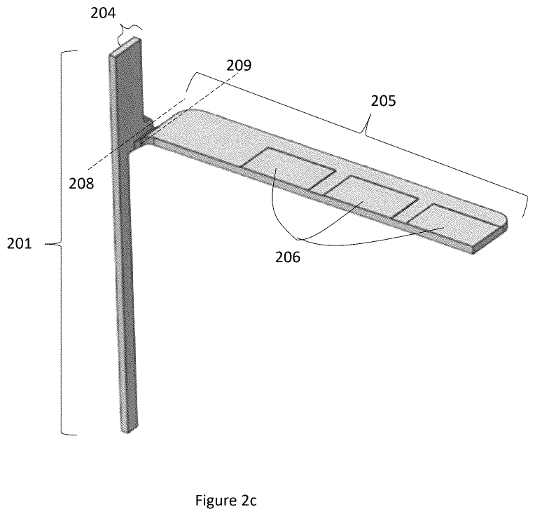

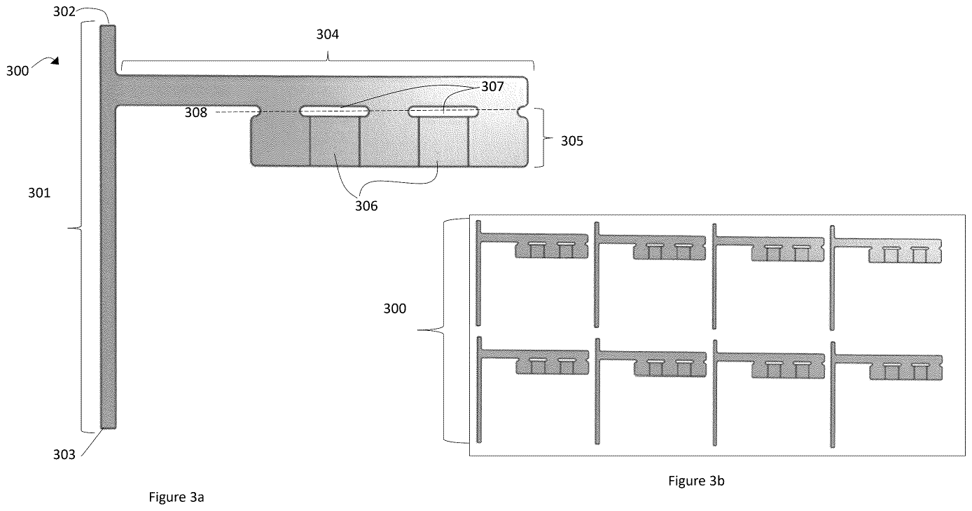

FIG. 2a illustratively shows an analyte sensor 200 according to the present disclosure prior to use where all the portions of the analyte sensor are provided in a plane as a result of being manufactured by stamping or cutting from a single sheet of material, for example plastic. The analyte sensor comprises a tail portion 201 having a proximal end 202 and a distal end 203. The distal end 203 is configured for insertion into the skin of a user, for example with the help of a sharp such as a cannula as will be appreciated by the skilled person.

The analyte sensor 200 comprises a lateral portion 204 extending in a lateral direction from the tail portion 201 in the plane of the analyte sensor 200, that is, the plane defined by the tail portion 201 and the lateral portion 204. It will accordingly be understood that the lateral direction is generally perpendicular to the length of the tail portion 201 between the proximal end 202 and distal end 203. The lateral portion 204 functions as a stability portion of the analyte sensor 200, that is, it may extend into a channel of a cannula used to insert the analyte sensor 200 into the skin to prevent the analyte sensor 200 from rotating in the cannula during insertion into the skin. It may also be held securely in a channel of a sensor module after insertion into the skin to stabilise the sensor and prevent the sensor from rotating or moving in the sensor module. The lateral portion 204 accordingly allows the tail to bend while also staying in a stable position.

The analyte sensor 200 further comprises a contact portion 205 having one or more conductive contact pads 206 thereon that function as electrodes and are configured for contact with a corresponding contact pad on a printed circuit board (PCB) of a sensor module of an analyte monitoring system (not shown). Whilst not shown, the one or more contact pads 206 are connected by one or more traces that extend along the contact portion 205 and the lateral portion 204 to a sensing layer in the tail portion 201. The sensing layer may comprise an electron transfer agent but other sensing layers for implantable analyte sensors known to the skilled person are also envisaged.

The analyte sensor 200 is configured to be folded or bent one or more times along a line 208 between the lateral portion 204 and the contact portion 205 so that the contact portion 205 can be folded or bent out of the plane and made perpendicular to the plane which is the configuration the analyte sensor 200 is in during use.

The contact pads 206 are provided along an edge of the contact portion 205. As described above, this allows the edge of the contact portion to be used as an alignment guide when placing the analyte sensor 200 in a sensor module to ensure the contact pads 206 align with and contact corresponding contact pads on the PCB. This ensures that the electrical connection between the analyte sensor 200 and the PCB is between the intended contact pads and reduces the risk of any short-circuits occurring from contact between the wrong pads due to misalignment. In the example of FIG. 2a, the contact pads 206 are provided along the edge closest to the tail portion and spaced out in a row in the distal direction, parallel to the tail portion 201.

The lateral portion 204 in FIG. 2a further comprises a shoulder 207 that extends from a distal end of the lateral portion 204 in the lateral direction in the plane of the analyte sensor 200. This allows the contact portion 205 to be shifted a predetermined distance away from the tail portion 201 to reduce the risk that stamping or cutting, or indeed depositing conductive material of the contact pads during manufacture, damages the tail portion 201, contact portion 205, contact pads 206 on the contact portion 205, or other parts of the sensor 200. The shoulder 207 where it joins the contact portion 205 is narrower than the lateral portion which facilitates easier bending or folding of the analyte sensor. Thus, synergistically, the combination of the lateral portion 204 acting as a stability portion and the shoulder 207 being narrow provides an analyte sensor that remains stable during insertion with a cannula and during use in a sensor module, while also being easy to bend during manufacture. Further, as stability is provided by the lateral portion, it is not necessary for the tail portion 201 itself to have any stability mechanisms or features with its engagement with the cannula, as a result, the cannula can encase the entire tail portion, leaving only the lateral portion exposed outside of the cannula during insertion.

The contact portion 205 extends in a distal direction from the lateral portion 204 and this means the total footprint or length of the analyte sensor 200 in the proximal direction is substantially reduced compared to for example if the contact portion 205 extends in the proximal direction, beyond the proximal end of the tail portion 201, and/or is wider in the lateral direction 200 than the sensor shown in FIG. 2a. In other words, the maximal extent of the analyte sensor in the proximal direction is determined by the position of the proximal edge of the proximal end of the tail portion 201. In the case of FIG. 2a, the proximal edge of the lateral portion 204 is in line with and extends from the corresponding proximal edge of the proximal end of the tail portion 201. This ensures there is no proximally protruding tail portion section which is thin and thus easily broken or snapped and thus further reduces the risk of accidental breakage of the analyte sensor during use. This configuration with a small footprint also provides a particular advantage when manufacturing the analyte sensor 200 in bulk as it is easier to fit many more of the small footprint analyte sensors 200 in a single row on a plastic sheet. This reduces the overall cost of manufacturing and reducing waste plastic as a larger area of the plastic sheet used during manufacture is being used.

FIG. 2b illustratively shows a plurality of analyte sensors 200 such as those of FIG. 2a arranged in the pattern they would be provided in on a plastic sheet during manufacture. It is apparent from FIG. 2b that there is very little wasted, unused space between the analyte sensors 200 as a result of the small footprint of the analyte sensor 200. As a result, the amount of plastic waste generated during manufacture is reduced. The array shown in FIG. 2b has five analyte sensors 200 per row and there are five rows, however it is envisaged that other patterns and arrangements may also be used.

FIG. 2c illustratively shows the analyte sensor of FIG. 2a after it has been bent of folded along line 208, thereby moving the contact portion 205 out of the plane defined by the tail portion 201 and lateral portion 204 and thus made perpendicular to that plane so that it is in the configuration it will be in during use. In the example of FIG. 2c, an additional fold is made in the contact portion along line 209 so that the contact portion 205 has an S-like bend in it. This ensures that an initial part of the bend in the contact portion 205 is directed in the distal direction and minimises the risk that a force applied to the analyte sensor 200 during insertion into the skin inadvertently over-bends the analyte sensor 200 while it is held in a sensor module during insertion.

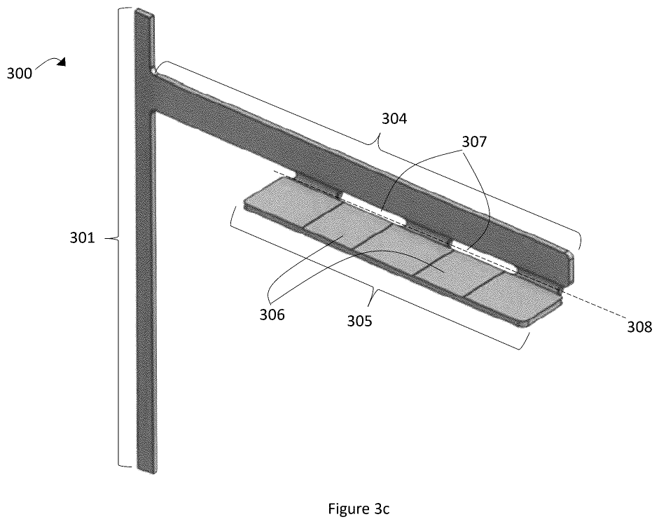

FIG. 3a illustratively shows an analyte sensor 300 according to the present disclosure prior to use where all the portions of the analyte sensor 300 are provided in a plane as a result of being manufactured by stamping or cutting from a single sheet of material, for example plastic. The analyte sensor 300 comprises a tail portion 301 having a proximal end 302 and a distal end 303. The distal end 303 is configured for insertion into the skin of a user, for example with the help of a sharp such as a cannula as will be appreciated by the skilled person.

The analyte sensor 300 comprises a lateral portion 304 extending in a lateral direction from the tail portion 301 in the plane of the analyte sensor 300, that is, the plane defined by the tail portion 301 and the lateral portion 304. As in FIG. 2a, the lateral portion 304 in FIG. 3a functions as a stability portion of the analyte sensor 300, that is, it may extend into a channel of a cannula used to insert the analyte sensor 300 into the skin to prevent the analyte sensor 300 from rotating in the cannula during insertion into the skin. It may also be held securely in a channel of a sensor module after insertion into the skin to stabilise the sensor and prevent the sensor from rotating or moving in the sensor module. The lateral portion 304 accordingly allows the tail to bend while also staying in a stable position. The lateral portion in FIG. 3a extends substantially further out from the tail portion 301 than in FIG. 2a. For example, the extent of the lateral portion 304 in the lateral direction is greater than the extent of the contact portion 305 in the lateral direction. This has three advantages. Firstly, given the large lateral extent of the lateral portion, the lateral portion is better able to resist any rotation, both about the axis of the tail portion but also about an axis of rotation perpendicular to the tail portion. Secondly, it even further reduces the risk that any stray conductive material is deposited on the tail portion 301 during manufacture of the contact pads of the contact portion, and reduces the risk of damage to the contact portion during stamping or cutting around the tail portion. Thirdly, it allows the contact portion and lateral portion 304 to share a much longer edge or join, thereby facilitating the introduction of perforations 307 between the contact portion and the lateral portion 305—something which is not possible to do where the join between the contact portion and lateral portion 304 is thin, as it is in FIG. 2a. This is because perforating an already thin join, makes it far too likely to break during bending or folding. Thus, the longer shared edge or join provides a stronger link that reduces the risk of breaking during bending or folding of the analyte sensor 300 while the perforations 307 advantageously make it easier to bend the now stronger link.

As with FIG. 2a, the analyte sensor 300 of FIG. 3a further comprises the above described contact portion 305 joined to the lateral portion along a shared border that it at least as long as the length of the contact portion 305 in the lateral direction. As described above, the contact portion 305 has one or more conductive contact pads 306 thereon that function as electrodes and are configured for contact with a corresponding contact pad on a printed circuit board (PCB) of a sensor module of an analyte monitoring system (not shown). Whilst not shown, the one or more contact pads 306 are connected by one or more traces that extend along the contact portion 305, between the perforations to the lateral portion 304 and finally to a sensing layer in the tail portion 301. The sensing layer may comprise an electron transfer agent but other sensing layers for implantable analyte sensors known to the skilled person are also envisaged.

The analyte sensor 300 is configured to be folded or bent one or more times along a line 308, that is, along the perforations 307 between the lateral portion 304 and the contact portion 305 so that the contact portion 305 can be folded or bent out of the plane and made perpendicular to the plane which is the configuration the analyte sensor 300 is in during use. In FIG. 3a, the contact portion 305 extends from a distal edge of the lateral portion 304.

The contact pads 306 are provided along an edge of the contact portion 305. As described above, this allows the edge of the contact portion to be used as an alignment guide when placing the analyte sensor 300 in a sensor module to ensure the contact pads 306 align with and contact corresponding contact pads on the PCB. This ensures that the electrical connection between the analyte sensor 300 and the PCB is between the intended contact pads and reduces the risk of any short-circuits occurring from contact between the wrong pads due to misalignment. In the example of FIG. 3a, the contact pads 306 are provided along the distal edge of the contact portion, spaced out in a row in the lateral direction.

As with FIG. 2a, the contact portion 305 in FIG. 3a extends in a distal direction from the lateral portion 304 and this means the total footprint or length of the analyte sensor 300 in the proximal direction is substantially reduced compared to for example if the contact portion 305 extends in the proximal direction, beyond the proximal end of the tail portion 301. In other words, the maximal extent of the analyte sensor 300 in the proximal direction is determined by the position of the proximal edge of the proximal end of the tail portion 301.

Unlike in FIG. 2a, the proximal edge of the lateral portion 304 of FIG. 3a is not in line with the proximal end of the tail portion but instead extends from the tail portion a predetermined distance down the tail portion 301.

FIG. 3b illustratively shows a plurality of analyte sensors 300 such as those of FIG. 3a arranged in the pattern they would be provided in on a plastic sheet during manufacture. The array shown in FIG. 3b has four analyte sensors 300 per row and there are two rows, however it is envisaged that other patterns and arrangements may also be used.

FIG. 3c illustratively shows the analyte sensor of FIG. 3a after it has been bent of folded along line 308 along the perforations 307, thereby moving the contact portion 305 out of the plane defined by the tail portion 301 and lateral portion 304 and thus made perpendicular to that plane so that it is in the configuration it will be in during use.

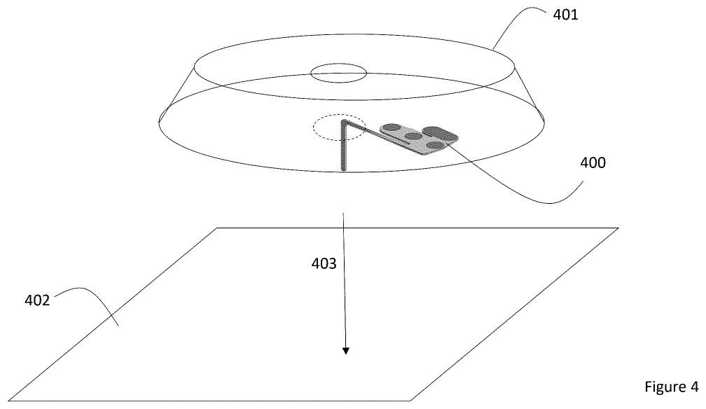

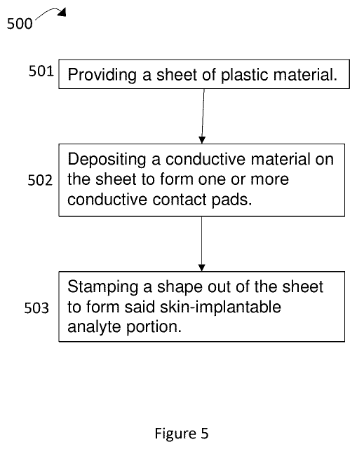

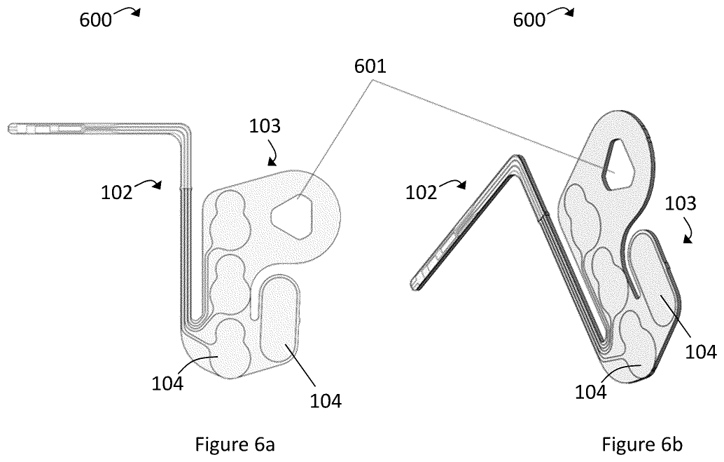

FIG. 4 illustratively shows an analyte sensor 400, such as that of FIG. 2c or FIG. 3c, positioned in a sensor module 401 of an analyte monitoring system. The sensor module 401, whilst not shown, comprises suitable circuitry to read signals from the analyte sensor 400 and record these and/or transmit them to a user interface, for example on a user's smartphone. The analyte sensor 400 protrudes from an opening in a distal surface of the sensor module and is configured to be inserted into a user's skin 402, as is indicated by arrow 403. The sensor module 401 is configured to be placed on top of the skin surface and secured there to protect the inserted part of the analyte sensor 402 until it needs to be replaced.

FIG. 5 illustratively shows a flowchart of a method 500 of the present disclosure. The method comprises: providing 501 a sheet of plastic material; depositing 502 a conductive material on the sheet to form one or more conductive contact pads; and stamping 503 a shape out of the sheet to form said skin-implantable analyte portion. It is envisaged that the shape corresponds to the shape of the analyte sensors described above in connection with FIGS. 1a-2c. Further, it is envisaged that the final step of the method may comprise stamping a plurality of said shapes out of the sheet of material, the shapes being arranged in a layout accordance with FIG. 2b or 2c, thereby providing an efficient method of manufacturing such analyte sensors in high volume (e.g. per sheet/row) with reduced wastage.

FIG. 6a and b show views of a further example analyte sensor 600. Various features of analyte sensor 600 correspond to previously discussed implementations, and like reference numerals are provided.

Analyte sensor 600 additionally comprises an aperture, opening or eyelet 601. The eyelet 600 may assist with correctly positioning the analyte sensor during the constructions of a sensing module, and further in securing the analyte sensor within e.g. a housing of the sensing module both post-manufacture and during use. For example, the housing of the sensing module may comprise a corresponding post or protrusion configured for insertion into the eyelet 601 of the analyte sensor 600. The eyelet 601 and protrusion may have corresponding shapes. While a triangular eyelet is depicted, it will be understood that any other shapes may also be provided for the eyelet, including but not limited to a circle, oval, ellipse, triangle, rectangle, diamond, pentagon, etc. Advantageously, shapes with one or more corners (such as triangles) may be particularly effective at maintaining a suitable position for the analyte sensor during construction of a corresponding sensing module. Optionally, the eyelet 601 may be provided with an irregular shape, such that the eyelet 601 can only receive the corresponding protrusion when the analyte sensor 600 is in the desired orientation.

It will be understood that such an opening or eyelet 600 may be incorporated into any of the analyte sensors implementations discussed herein.

Other effective alternatives will occur to the skilled person. It will be understood that the invention is not limited to the described embodiments and encompasses modifications apparent to those skilled in the art lying within the scope of the claims appended hereto.

For example, the term distal refers to a direction towards a user's skin surface when the analyte sensor is in use and may also be described as the forward direction. Conversely, proximal refers to a direction away from a user's skin surface when the analyte sensor is in use and may also be described as the rearward direction.

For example, the term contact pad may refer to any area or deposit of conductive material including for example one or more of the following non-limiting examples: solder material and/or gold trace.

For example, the advantages of the twisted portion may be provided on any lateral portion that is long enough to sustain a 90 degree twist without snapping.

For example, the deformable finger portion described above may have any number of contact portions provided thereon to facilitate the switch-on mechanism described above.

For example, whilst the present disclosure describes providing a contact portion that extends in a distal direction from the lateral portion, it is also envisaged that the advantage of providing perforations between the lateral portion and the contact portion may be provided with the contact portion extending in a proximal direction from the lateral portion instead. In this case, a specific advantage provided is that the contact portion is easier to fold without breaking compared to known systems, such as that of EP2393417B1. Thus, there is envisaged a skin-implantable analyte sensor for use with an analyte monitoring system, the analyte sensor comprising: a tail portion configured for insertion into a user's skin, the tail portion having a proximal end and a distal end; a lateral portion extending in a lateral direction from the tail portion, the tail portion and the lateral portion defining a plane; and a contact portion having one or more conductive contact pads thereon, wherein, prior to use, the contact portion is in the plane and extends from the lateral portion in a proximal direction towards the proximal end, wherein, during use, the contact portion extends from the lateral portion in a direction out of and perpendicular to the plane, and wherein the analyte sensor comprises one or more perforations between the lateral portion and the contact portion. Optionally, a length of the lateral portion in the lateral direction is greater than a length of the contact portion in the lateral direction.

1. A skin-implantable analyte sensor for use with an analyte monitoring system, the analyte sensor comprising:
a tail portion configured for insertion into a user's skin, the tail portion having a proximal end and a distal end;
a lateral portion extending in a lateral direction from the tail portion, the tail portion and the lateral portion defining a plane; and
a contact portion having one or more conductive contact pads thereon,
wherein, during use, the contact portion extends from the lateral portion in a direction out of and perpendicular to the plane;
wherein a length of the lateral portion in the lateral direction is greater than a length of the contact portion in the lateral direction.

2. The skin-implantable analyte sensor of claim 1, wherein, during use, the lateral portion comprises a twisted section; and optionally wherein the twisted section comprises a 90 degree twist.

3. (canceled)

4. The skin-implantable analyte sensor of claim 1, comprising one or more perforations between the lateral portion and the contact portion.

5. The skin-implantable analyte sensor of claim 4, wherein, prior to use, the contact portion is configured to be folded relative to the lateral portion along the one or more perforations.

6. The skin-implantable analyte sensor of claim 4, wherein, during use, a length of the lateral portion in the proximal direction is greater than a thickness of the contact portion in the proximal direction.

7. The skin-implantable analyte sensor of claim 4, wherein the conductive contact pads are provided at an edge of the contact portion; and optionally wherein said edge is parallel to the lateral direction.

8. (canceled)

9. The skin-implantable analyte sensor of claim 1, wherein the contact portion comprises a deformable finger portion having at least one of said conductive contact pads thereon.

10. The skin-implantable analyte sensor of claim 1, wherein the tail portion comprises a sensing layer electrically coupled to the one or more conductive contact pads by one or more conductive traces extending along a surface of the tail portion, a surface of the lateral portion, and a surface of the contact portion.

11. The skin-implantable analyte sensor of claim 1, wherein, prior to use, at least one of:
(i) the contact portion is in the plane and extends from the lateral portion in a proximal direction away from the proximal end; and
(ii) the contact portion is in the plane and extends from the lateral portion in a distal direction towards the distal end.

12. (canceled)

13. The skin-implantable sensor of claim 1, wherein the lateral portion extends from the tail portion at the proximal edge of the proximal end of the tail portion in the plane.

14. The skin-implantable analyte sensor of claim 1, configured as a blood-glucose analyte sensor.

15. The skin-implantable analyte sensor of claim 1, further comprising an aperture configured to positionally secure the analyte sensor within an analyte monitoring system; and optionally wherein a shape of the aperture comprises one or more corners, and optionally wherein aperture is triangular.

16. (canceled)

17. A skin-implantable analyte sensor for use with an analyte monitoring system, the analyte sensor comprising:
a tail portion configured for insertion into a user's skin, the tail portion having a proximal end and a distal end;
a lateral portion extending in a lateral direction from the tail portion, the tail portion and the lateral portion defining a plane; and
a contact portion having one or more conductive contact pads thereon,
wherein, prior to use, the contact portion is in the plane and extends from the lateral portion in a distal direction towards the distal end,
wherein, during use, the contact portion extends from the lateral portion in a direction out of and perpendicular to the plane.

18. The skin-implantable analyte sensor of claim 17, wherein the lateral portion comprises a shoulder portion at a lateral edge,
wherein, prior to use, the contact portion extends in a distal direction from a distal edge of the shoulder portion of the lateral portion, and
wherein, the analyte sensor is configured to be folded along a line between the shoulder portion and the contact portion.

19. The skin-implantable analyte sensor of claim 17 configured as a blood-glucose analyte sensor.

20. The skin-implantable analyte sensor of claim 17, wherein the contact portion comprises a deformable finger portion having at least one of said conductive contact pads thereon.

21. The skin-implantable analyte sensor of claim 17, further comprising an aperture configured to positionally secure the analyte sensor within an analyte monitoring system; and optionally wherein a shape of the aperture comprises one or more corners, and optionally wherein aperture is triangular.

22. (canceled)

23. A method of manufacturing a skin-implantable analyte sensor of any preceding claim, the method comprising:
providing a sheet of plastic material;
depositing a conductive material on the sheet to form one or more conductive contact pads; and
stamping a shape out of the sheet to form said skin-implantable analyte portion, the shape comprising:
a tail portion having a proximal end and a distal end;
a lateral portion extending in a lateral direction from the tail portion, the tail portion and the lateral portion defining a plane; and
a contact portion having said one or more conductive contact pads thereon,
wherein a length of the lateral portion in the lateral direction is greater than a length of the contact portion in the lateral direction.

24. The method of claim 23, comprising twisting the lateral portion to cause the contact portion to extend out of the plane.

25. The method of claim 23, comprising perforating the analyte sensor between the lateral portion and the contact portion; and optionally comprising folding the analyte sensor along the perforations to cause the contact portion to extend out of the plane.

26. (canceled)
